(19) United States
(12) Patent Application Publication
(10) Pub. No.: US 2019/0307332 A1
(43) Pub. Date: Oct. 10, 2019

(54) SOLID SAMPLE FOR CALIBRATION, ENDOSCOPE SYSTEM, AND PREPARATION METHOD OF SOLID SAMPLE

(71) Applicant: HOYA CORPORATION, Tokyo (JP)

(72) Inventor: Toru CHIBA, Tokyo (JP)

(73) Assignee: HOYA CORPORATION, Tokyo (JP)

(21) Appl. No.: 16/468,972

(22) PCT Filed: Jan. 9, 2018
(86) PCT No.: PCT/JP2018/000212

§ 371 Date: Jun. 12, 2019

(30) Foreign Application Priority Data
Jan. 6, 2017 (JP) 2017-000939

(51) Int. Cl. A61B 5/00; A61B 8/13; A61B 1/04; A61B 1/06; A61B 1/00

(52) U.S. Cl. CPC A61B 5/0084; A61B 8/13; G06T 2207/10068; A61B 1/0676; A61B 1/00009; A61B 1/04

(57) ABSTRACT

A solid sample to be used as a calibration reference sample to calculate a hemoglobin concentration and a hemoglobin oxygen saturation in a living tissue is made of non-biological substance having: a colorant group that has a plurality of colorants of non-biological substances and reproduces absorption characteristics of the hemoglobin with a predetermined concentration and a predetermined oxygen saturation by adjusting a mixing ratio of the plurality of colorants; and a resin material in which each colorant of the colorant group is dispersed. In preparing the solid sample, the colorant group reproducing the hemoglobin absorption characteristics with the predetermined hemoglobin concentration and the predetermined hemoglobin oxygen saturation is prepared, and then, resin as a base material is dissolved in a mixed solution in which the colorant group is dispersed in an organic solvent. The organic solvent is volatilized from the mixed solution in which the resin has been dissolved.

### TECHNICAL FIELD

The present invention relates to a solid sample made of a non-biological substance, which is used as a calibration reference sample of an endoscope system, the endoscope system, and a preparation method of the solid sample.

### BACKGROUND ART

There are known endoscope systems having a function of obtaining information on a biological substance in a living tissue, which is a subject, for example, a hemoglobin concentration or a hemoglobin oxygen saturation, from image data obtained by an endoscope and displaying the information as an image. Patent Literature 1 describes an example of a hemoglobin observation apparatus including such an endoscope system.

The hemoglobin observation apparatus described in Patent Literature 1 has a configuration in which, when a wavelength at which an absorption spectrum of oxyhemoglobin which has been bound with oxygen at 100% intersects an absorption spectrum of reduced hemoglobin from which oxygen has been released at 100% is set to an isoabsorption wavelength, an object to be observed including hemoglobin is irradiated with at least two different light of a first wavelength and light of a second wavelength in a wavelength range including the isoabsorption wavelength, an image of the object to be observed is captured based on reflected or transmitted light of the irradiation light, a predetermined operation is performed based on a signal of the captured image, and then, a result of the processing is displayed on a display unit. At this time, a binding state between hemoglobin and oxygen is calculated in the operation processing of the signal of the captured image based on a difference between a first reflected light amount or transmitted light amount of the light of the first wavelength and a second reflected light amount or transmitted light amount of the light of the second wavelength.

### CITATION LIST

### Patent Literature

Patent Literature 1: JP 2005-326153 A

### SUMMARY OF INVENTION

### Technical Problem

In the hemoglobin observation apparatus, an oxygen saturation is calculated using a ratio obtained by normalizing a difference between a first absorbance value O1 at the first wavelength indicating the amount of oxyhemoglobin and a second absorbance value O2 at the second wavelength indicating the amount of reduced hemoglobin.

However, a relationship between the first absorbance value O1 and a value of a signal at the first wavelength obtained by the hemoglobin observation apparatus or a relationship between the second absorbance value O2 and a value of a signal at the second wavelength obtained by the hemoglobin observation apparatus varies as an error between hemoglobin observation apparatuses, and often changes over time with the long-term use of the apparatus even if the single hemoglobin observation apparatus is used. In addition, a correction coefficient is often used so as to make a value of the above ratio match an intermediate oxygen saturation between 0% and 100%.

Thus, in order to calculate a hemoglobin oxygen saturation with high accuracy, it is preferable to actually observe oxyhemoglobin and reduced hemoglobin and associate calculation results relating to hemoglobin obtained from the observation with information on the actual concentration and oxygen saturation of the observed oxyhemoglobin in the endoscope system. For example, it is preferable to obtain a correspondence relationship between data corresponding to the concentration of the oxyhemoglobin or data corresponding to the oxygen saturation of the oxyhemoglobin obtained from the observation by the endoscope system and a value of the actual concentration of the observed oxyhemoglobin or oxygen saturations of the oxyhemoglobin and reduced hemoglobin in advance, and to obtain the amount of oxyhemoglobin and an oxygen saturation of a living tissue, set as an actual observation target, using this correspondence relationship.

The correspondence relationship is created, for example, at the time of completion of the endoscope system using a reference sample having a predetermined hemoglobin concentration and a predetermined hemoglobin oxygen saturation, and is recorded and held in the endoscope system. However, the relationship changes over time with the use of the endoscope system as described above, and thus, it is preferable to reset the above correspondence relationship by performing calibration to calculate an oxygen saturation just before an observation whenever a living tissue is observed by the endoscope system to calculate the hemoglobin oxygen saturation with high accuracy. A calibration reference sample is used for such resetting. For example, a biological substance such as hemoglobin is used as the calibration reference sample. However, bringing such a calibration reference sample made of the biological substance into a medical facility or a medical site is difficult and restricted from the viewpoint of safety. In addition, reduced hemoglobin, which is used as the calibration reference sample, is an unstable substance which is likely to be in contact with oxygen to form oxyhemoglobin. Thus, it is desirable to use a calibration reference sample made of a stable non-biological substance simulating hemoglobin, instead of the calibration sample made of the biological substance. However, the calibration reference sample that is made of the non-biological substance and stable without change in oxygen saturation is not currently known.

Therefore, an object of the present invention is to provide a stable solid sample made of a non-biological substance capable of calibration, instead of a calibration reference sample made of a biological substance, an endoscope system that performs calibration using the solid sample, and, a preparation method of the solid sample.

### Solution to Problem

One aspect of the present invention is a solid sample.

The solid sample includes: a colorant group made of non-biological substances which have a plurality of colorants and reproduce absorption characteristics of the hemoglobin with a predetermined concentration and a predetermined oxygen saturation by adjusting a mixing ratio of the plurality of colorants; and a resin material in which each colorant of the colorant group is dispersed, and is made of a non-biological substance.

The solid sample is used as a calibration reference sample to calculate a hemoglobin concentration and a hemoglobin oxygen saturation in a living tissue.

Another aspect of the present invention is a method of calculating a hemoglobin concentration and a hemoglobin oxygen saturation in a living tissue using an endoscope system, or use of a solid sample which is used for calculation of a hemoglobin concentration and the hemoglobin oxygen saturation in the living tissue using an endoscopic system.

The endoscope system includes: an endoscope including an imaging unit provided with an imaging element configured to image the living tissue to generate a plurality of pieces of image data; and a processor configured to calculate values of a first ratio and a second ratio between predetermined components using values of the components out of components of the plurality of pieces of image data and calculate the hemoglobin concentration and the hemoglobin oxygen saturation in the living tissue using the values of the first ratio and the second ratio.

The processor stores a first correspondence relationship including a first association between a calibration measurement value of the first ratio, which is a measurement result imaged by the endoscope using the above solid sample as a calibration reference sample for calculation of the hemoglobin oxygen saturation, and information on the predetermined hemoglobin concentration in the solid sample, and a second correspondence relationship including a second association between a calibration measurement value of the second ratio, which is a measurement result imaged by the endoscope using the solid sample as the calibration reference sample, and information on the predetermined hemoglobin oxygen saturation in the solid sample in a storage unit, and the processor refers to the first correspondence relationship and the second correspondence relationship using the values of the first ratio and the second ratio to calculate the hemoglobin concentration and the saturation oxygen saturation in the living tissue.

Yet another aspect of the present invention is a method of calculating a hemoglobin concentration and a hemoglobin oxygen saturation in a living tissue using an endoscope system, or use of a solid sample which is used for calculation of a hemoglobin concentration and a hemoglobin oxygen saturation in a living tissue using an endoscopic system.

The endoscope system includes: an endoscope including an imaging unit provided with an imaging element configured to image the living tissue to generate a plurality of pieces of image data; and a processor configured to calculate values of a first ratio and a second ratio between predetermined components using values of the components out of components of the plurality of pieces of image data and calculate the hemoglobin concentration and the hemoglobin oxygen saturation in the living tissue using the values of the first ratio and the second ratio.

The processor stores, in a storage unit, a first correspondence relationship between the hemoglobin concentration and the value of the first ratio, a second correspondence relationship between the hemoglobin oxygen saturation and the value of the second ratio, and correction coefficients which allow a calibration measurement value of the first ratio and a calibration measurement value of the second ratio, which are measurement results obtained by imaging the above solid sample, as a calibration reference sample for calculation of the hemoglobin oxygen saturation, with the endoscope, to be corrected to preset values, respectively. The processor refers to the first correspondence relationship and the second correspondence relationship using the values, obtained by correcting the values of the first ratio and the second ratio obtained using a value of the image data, using the correction coefficients to calculate the hemoglobin concentration and the hemoglobin oxygen saturation in the living tissue.

Each of the above aspects includes the following preferred mode.

In the solid sample, the colorant group preferably includes at least a first colorant having two absorption peak wavelengths in a wavelength band where a wavelength is 520 to 600 nm, and a second colorant having one absorption peak wavelength in a wavelength band where a wavelength is 400 to 440 nm, and a wavelength band of the absorption characteristics reproduced by the colorant group is preferably a wavelength band of 400 to 600 nm.

It is preferable that an absorption spectrum of the wavelength band of 520 to 600 nm in the solid sample have two absorption peaks and an absorption bottom which is sandwiched between the two absorption peaks and has a lowest absorbance between the two absorption peaks, that each wavelength deviation between each of the two absorption peaks and each of corresponding absorption peaks of the hemoglobin, which respectively correspond the two absorption peaks, be 2 nm or less, that a wavelength deviation between the absorption bottom and a corresponding absorption bottom of the hemoglobin, which corresponds to the absorption bottom, be 2 nm or less, and that each absorbance at each of the two absorption peaks be in a range of 95% to 105% with respect to each absorbance at each of the corresponding absorption peaks of the hemoglobin which respectively correspond to the two absorption peaks.

In addition, it is also preferable that an absorption spectrum of the wavelength band where the wavelength is 520 to 600 nm in the solid sample have one absorption peak in a range of 546 to 570 nm, and that an absorbance at the absorption peak be in a range of 95% to 105% with respect to an absorbance at a corresponding absorption peak of the hemoglobin which corresponds to the absorption peak.

A variation depending on a place of the solid sample, of an average absorbance in the wavelength band where the wavelength is 520 to 600 nm of the solid sample is preferably 5% or less of an average value of the average absorbances for the place.

A variation depending on a place of the solid sample, of a ratio of an average absorbance in a wavelength band where a wavelength is 546 to 570 nm relative to an average absorbance in a wavelength band where a wavelength is 528 to 584 nm of the solid sample is preferably 1% or less of an average value of the ratios for the place.

Another aspect of the present invention is an endoscope system. The endoscope system includes: an endoscope including an imaging unit which has an imaging element configured to generate a plurality of pieces of image data by imaging a living tissue; and a processor configured to calculate values of a first ratio and a second ratio between predetermined components using values of the components out of components of the plurality of pieces of image data and to calculate a hemoglobin concentration and a hemoglobin oxygen saturation in a living tissue using the values of the first ratio and the second ratio. The processor includes a storage unit storing a first correspondence relationship between the hemoglobin concentration and the value of the first ratio, the first correspondence relationship including an association between a calibration measurement value of the first ratio, which is a measurement result obtained by imaging the solid sample with the endoscope as a calibration reference sample for calculation of the hemoglobin oxygen saturation, and information on the predetermined hemoglobin concentration of the solid sample, and a second correspondence relationship between the hemoglobin oxygen saturation and the value of the second ratio, the second correspondence relationship including an association between a calibration measurement value of the second ratio, which is a measurement result obtained by imaging the solid sample with the endoscope, as the calibration reference sample, and information on the predetermined hemoglobin oxygen saturation of the solid sample.

The processor is configured to calculate the hemoglobin concentration and the hemoglobin oxygen saturation in the living tissue using the first correspondence relationship and the second correspondence relationship.

Another aspect of the present invention is an endoscope system.

The endoscope system includes: an endoscope including an imaging unit which has an imaging element configured to generate a plurality of pieces of image data by imaging a living tissue; and a processor configured to calculate values of a first ratio and a second ratio between predetermined components using values of the components out of components of the plurality of pieces of image data and to calculate a hemoglobin concentration and a hemoglobin oxygen saturation in a living tissue using the values of the first ratio and the second ratio. The processor includes a storage unit storing a first correspondence relationship between the hemoglobin concentration and the value of the first ratio, a second correspondence relationship between the hemoglobin oxygen saturation and the value of the second ratio, and correction coefficients which allow a calibration measurement value of the first ratio and a calibration measurement value of the second ratio, which are measurement results obtained by imaging the above solid sample, as a calibration reference sample for calculation of the hemoglobin oxygen saturation, with the endoscope, to be corrected to preset values, respectively. The processor is configured to refer to the first correspondence relationship and the second correspondence relationship using the values, obtained by correcting the values of the first ratio and the second ratio obtained using a value of the image data, using the correction coefficients to calculate the hemoglobin concentration and the hemoglobin oxygen saturation in the living tissue.

In the endoscope system, the calibration measurement value of the first ratio and the calibration measurement value of the second ratio are preferably measurement results obtained by imaging each of a plurality of types of solid samples having different contents of the colorant group corresponding to a plurality of hemoglobin concentrations, as the reference sample, with the endoscope.

It is preferable that the first ratio be a ratio sensitive to the hemoglobin concentration of the living tissue, that the second ratio be a ratio sensitive to the hemoglobin oxygen saturation of the living tissue, that one of the components of the image data used for calculation of the first ratio be a component of a first wavelength band within a range of 500 nm to 600 nm, and that one of the components of the image data used for calculation of the second ratio be a component of a second wavelength band narrower than the first wavelength band.

Yet another aspect of the present invention is a preparation method of a solid sample made of a non-biological substance, which is used as a calibration reference sample to calculate a hemoglobin oxygen saturation.

The preparation method includes: a step of preparing a colorant group reproducing an absorption characteristic of hemoglobin having a predetermined hemoglobin oxygen saturation; a step of dissolving resin as a base material in a mixed solution in which a predetermined amount of the colorant group for reproduction of an absorption characteristic of hemoglobin with a predetermined concentration is dispersed in an organic solvent; and a step of volatilizing the organic solvent from the mixed solution in which the resin has been dissolved to prepare the solid sample.

In the preparation method of the solid sample, it is preferable that the colorant group include at least a first colorant having two absorption peak wavelengths in a wavelength band where a wavelength is 520 to 600 nm, and a second colorant having one absorption peak wavelength in a wavelength band where a wavelength is 400 to 440 nm.

In addition, still yet another aspect of the present invention is a method of performing calibration of an endoscope and a processor in order to calculate a hemoglobin concentration and a hemoglobin oxygen saturation in a living tissue using the endoscope and the processor, the hemoglobin concentration and the hemoglobin oxygen saturation in a living tissue calculated using values of a first ratio and a second ratio between predetermined components, the first ratio and the second ratio calculated using values of the components out of components of a plurality of pieces of image data obtained by imaging the living tissue illuminated with a plurality of beams of light using the endoscope. The method of performing calibration includes: a step of imaging the solid sample with the endoscope to acquire each of a calibration measurement value of the first ratio and a calibration measurement value of the second ratio; a step of causing the processor to generate a first correspondence relationship between the hemoglobin concentration and the value of the first ratio, which includes a first association between the calibration measurement value of the first ratio and information on the predetermined hemoglobin concentration of the solid sample, and generate a second correspondence relationship between the hemoglobin oxygen saturation and the value of the second ratio, which includes a second association between the calibration measurement value of the second ratio and information on the predetermined hemoglobin oxygen saturation; and a step of causing the processor to store the first correspondence relationship and the second correspondence relationship in order to use the first correspondence relationship and the second correspondence relationship for calculation of the hemoglobin concentration and the hemoglobin oxygen saturation in the living tissue.

In addition, still yet another aspect of the present invention is a method of performing calibration of an endoscope and a processor in order to calculate a hemoglobin concentration and a hemoglobin oxygen saturation in a living tissue using the endoscope and the processor, the hemoglobin concentration and the hemoglobin oxygen saturation in a living tissue calculated using values of a first ratio and a second ratio between predetermined components, the first ratio and the second ratio calculated using values of the components out of components of a plurality of pieces of image data obtained by imaging the living tissue illuminated with a plurality of beams of light using the endoscope. The method of performing calibration includes: a step of imaging the above solid sample with the endoscope to acquire each of a calibration measurement value of the first ratio and a calibration measurement value of the second ratio; a step of causing the processor to calculate correction coefficients which allow the calibration measurement value of the first ratio and the calibration measurement value of the second ratio to be corrected to preset values, respectively; and a step of causing the processor to store the correction coefficients to correct the first ratio and the second ratio using the correction coefficients, respectively, in order to use the correction coefficients to calculate the hemoglobin concentration and the hemoglobin oxygen saturation in the living tissue.

In the method of performing the calibration, it is preferable that the solid sample include a plurality of types of samples having different contents of the colorant group corresponding to a plurality of hemoglobin concentrations and that the calibration measurement value of the first ratio and the calibration measurement value of the second ratio be measurement results obtained by imaging each of the plurality of types of samples as the reference sample with the endoscope.

It is preferable that the first ratio be a ratio sensitive to the hemoglobin concentration of the living tissue, that the second ratio be a ratio sensitive to the hemoglobin oxygen saturation of the living tissue, that one of the components of the image data used for calculation of the first ratio be a component of a first wavelength band within a range of 500 nm to 600 nm, and that one of the components of the image data used for calculation of the second ratio be a component of a second wavelength band narrower than the first wavelength band.

### Advantageous Effects of Invention

According to the above-described solid sample, it is possible to provide the stable sample made of the non-biological substance capable of the calibration, instead of the calibration reference sample made of the biological substance.

Therefore, it is possible to provide the endoscope system calibrated using the solid sample.

### BRIEF DESCRIPTION OF DRAWINGS

FIG. 1 is a view for describing an example of a calibration sample using a solid sample of the present embodiment.

FIG. 2 is a graph illustrating an example of absorption characteristics of the solid sample of the present embodiment.

FIGS. 3(a) and 3(b) are graphs illustrating examples of wavelength characteristics of optical densities of colorants used for the solid sample of the present embodiment.

FIG. 4 is a view for describing calibration of an endoscope system using the solid sample according to the present embodiment.

FIG. 5 is a block diagram of a configuration of an example of the endoscope system used in the present embodiment.

FIG. 6 is a graph illustrating an example of spectral characteristics of the respective filters of red (R), green (G) and blue (B) of an imaging element of the endoscope system used in the present embodiment.

FIG. 7 is an external view (front view) of an example of a rotary filter used in a light source device of the endoscope system used in the present embodiment.

FIG. 8 is a graph illustrating an example of an absorption spectrum of hemoglobin in the vicinity of 550 nm.

FIG. 9 is a graph illustrating an example of a relationship between a first ratio used in the present embodiment and a hemoglobin concentration.

FIG. 10 is a graph illustrating an example of a relationship between an upper limit value and a lower limit value of a second ratio used in the present embodiment, and the hemoglobin concentration.

### DESCRIPTION OF EMBODIMENTS

(Solid Sample)

A solid sample made of a non-biological substance according to the present embodiment described hereinafter is used as a calibration reference sample of an endoscope system configured to calculate a hemoglobin concentration in a living tissue and a hemoglobin oxygen saturation. The endoscope system used in the present embodiment is a system that quantitatively calculates a hemoglobin concentration in a living tissue and a hemoglobin oxygen saturation based on a plurality of pieces of color image data imaged by irradiating the living tissue, which is a subject, with beams of light in different wavelength ranges, and displaying a feature amount distribution image representing a distribution of the hemoglobin concentration or the hemoglobin oxygen saturation.

In the endoscope system, a parameter obtained from the image data of the living tissue imaged by the endoscope system is used to refer to a correspondence relationship between the hemoglobin concentration or the hemoglobin oxygen saturation and the parameter, thereby calculating the hemoglobin concentration or the hemoglobin oxygen saturation. The solid sample of the present embodiment is used to perform calibration in order to set the correspondence relationship at this time before using the endoscope system.

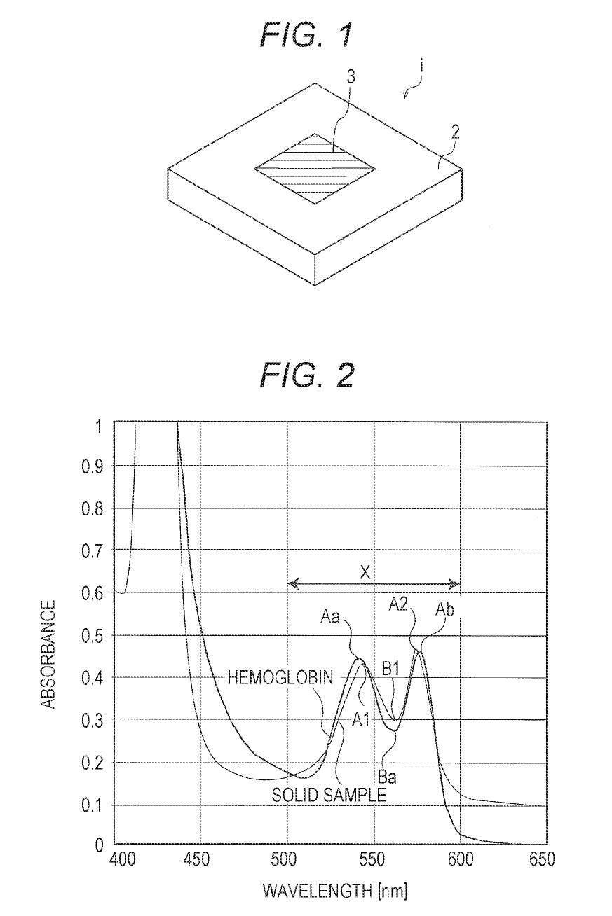

FIG. 1 is a view for describing an example of a calibration sample having the solid sample of the present embodiment. A calibration sample 1 is provided with a solid sample 3 on a base 2.

The base 2 is configured using a resin plate or a metal plate. The base 2 is preferably white.

The solid sample 3 is provided on a surface of the base 2.

The solid sample 3 is made of a non-biological substance and is not made of a biological substance such as blood.

The calibration sample 1 illustrated in FIG. 1 is a reflection-type sample that receives light, which has been transmitted through the solid sample 3 and been reflected on the surface of the base 2, by the endoscope system, but may be a transmission-type sample in which light having been transmitted through the solid sample 3 is received by the endoscope system.

The solid sample 3 is made of a plurality of types of colorants made of non-biological substances, and a resin material in which the plurality of types of colorants are dispersed. A mixing ratio of the plurality of types of colorants is adjusted such that the plurality of types of colorants reproduce absorption characteristics of hemoglobin at a predetermined hemoglobin concentration and a predetermined hemoglobin oxygen saturation. As the colorant of the solid sample 3, for example, a compound described in JP H2-196865 A can be used.

As a result, the absorption characteristics of the solid sample 3, that is, a spectral waveform of an absorbance substantially matches a spectrum waveform of an absorbance at the predetermined hemoglobin concentration and the predetermined hemoglobin oxygen saturation. FIG. 2 is a graph illustrating an example of the absorption characteristics of the solid sample 3 of the present embodiment.

Here, in a wavelength range X (500 nm to 600 nm), a spectrum waveform of the solid sample 3 substantially matches a spectrum waveform of an absorbance of oxyhemoglobin which is hemoglobin having an oxygen saturation of 100%. The wavelength range X is a wavelength range including a wavelength range R0 of image data of a living tissue imaged by an endoscope system 10, which is used at the time of obtaining the hemoglobin concentration and the hemoglobin oxygen saturation, to be described later.

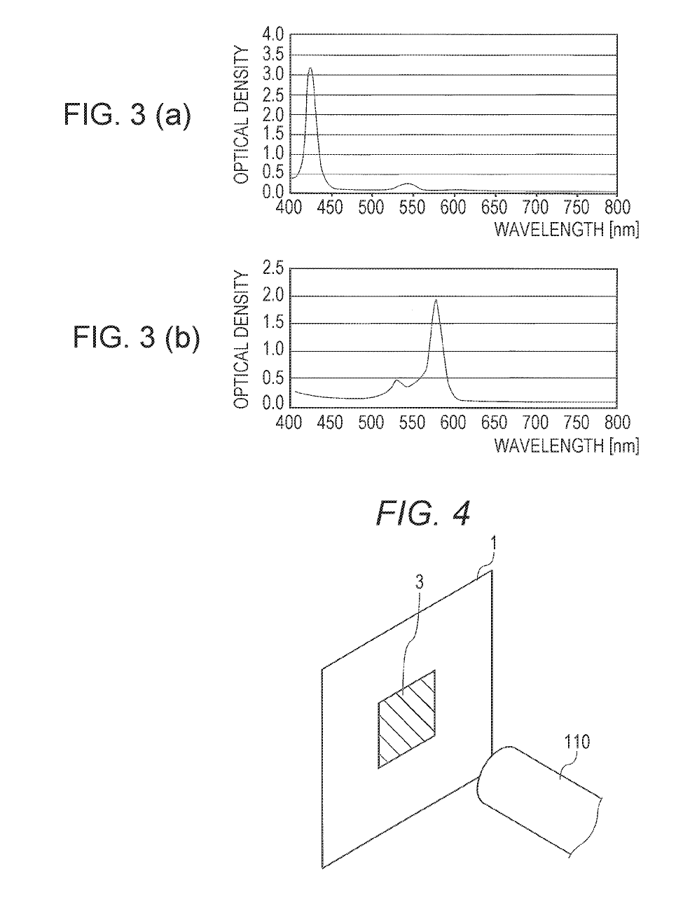

FIGS. 3(a) and 3(b) are graphs illustrating examples of wavelength characteristics of optical densities of the colorants used for the solid sample 3. The optical density reflects the absorption characteristic of light. The colorants used in the solid sample 3 are two types of colorants having the optical densities illustrated in FIGS. 3(a) and 3(b), respectively. As illustrated in FIG. 3(b), one colorant (a first colorant) has two peak wavelengths (absorption peak wavelengths) in a wavelength band where a wavelength is 520 to 600 nm. As illustrated in FIG. 3(a), another colorant (a second colorant) has one peak wavelength (absorption peak wavelength) in a wavelength band where a wavelength is 400 to 440 nm. When each content of the colorants is adjusted, it is possible to obtain a spectral wavelength of an absorption characteristic which substantially matches an absorption characteristic of hemoglobin in a wavelength band of 400 to 600 nm as illustrated in FIG. 2.

As illustrated in FIG. 2, an absorption spectrum of the wavelength band where the wavelength is 520 to 600 nm in the solid sample 3 of the present embodiment has two absorption peaks A1 and A2 and an absorption bottom B1 which is sandwiched between the two absorption peaks A1 and A2 and has a lowest absorbance between the two absorption peaks A1 and A2. Here, each wavelength deviation between each of the two absorption peaks A1 and A2 and each of corresponding absorption peaks Aa and Ab of hemoglobin, which respectively correspond the two absorption peaks A1 and A2, is preferably 2 nm or less, and more preferably, 1 nm or less. In addition, a wavelength deviation between the absorption bottom B1 and a corresponding absorption bottom Ba of hemoglobin, which corresponds to the absorption bottom B1, is preferably 2 nm or less, and more preferably 1 nm or less.

In addition, each absorbance at each of the two absorption peaks A1 and A2 is preferably 95% to 105%, and more preferably 97% to 103%, with respect to each absorbance at each of the corresponding absorption peaks Aa and Ab of hemoglobin which respectively correspond to the two absorption peaks A1 and A2. In addition, an absorbance at the absorption bottom B1 is preferably in the range of 95% to 105%, and more preferably in the range of 97% to 103%, with respect to an absorbance of the corresponding absorption bottom Ba of hemoglobin which corresponds to the absorption bottom B1.

Although the two types of colorants illustrated in FIGS. 3(a) and 3(b) are used as the colorant used for the solid sample 3, the number of types of colorants may be three or four. It is possible to make the absorption characteristic of the solid sample 3 match the absorption characteristic of hemoglobin by using these colorants.

Although not illustrated, a solid sample reproducing an absorption characteristic of hemoglobin having a different oxygen saturation may be prepared by adjusting each amount of the above two colorants. As a solid sample reproducing an absorption characteristic of reduced hemoglobin with an oxygen saturation of 0%, a solid sample having a different configuration from the solid sample 3 using the two types of colorants, for example, a compound having an absorption peak at 555 nm may be used.

In the present embodiment, calibration is performed using the solid sample 3 at least reproducing oxyhemoglobin having an oxygen saturation of 100% as illustrated in FIG. 2.

Since this solid sample 3 is the non-biological substance, the absorption characteristic is stable, and the absorbance changes little with time, which is different from the biological substance.

According to one embodiment, the above compound having the absorption peak at 555 nm is used, and thus, the absorption spectrum of the wavelength band of 520 to 600 nm in the solid sample 3 has one absorption peak in the range of 546 nm to 570 nm as in an absorption spectrum of reduced hemoglobin to be described later. In this case, an absorbance at the absorption peak in the range of 546 nm to 570 nm is preferably in the range of 95% to 105% with respect to an absorbance at a corresponding absorption peak of reduced hemoglobin which corresponds to the absorption peak.

This solid sample 3 can be prepared, for example, by the following method.

(1) A colorant group reproducing am absorption characteristic of hemoglobin having a predetermined hemoglobin oxygen saturation is prepared. The preparation of the colorant group includes selection of a plurality of types of colorant, adjustment of a mixing ratio of the selected colorants, and adjustment of the amount of the mixed colorant group. With the selection of the plurality of types of colorants and adjustment the mixing ratio of the selected colorants, it is possible to reproduce the absorption characteristic of hemoglobin having the predetermined oxygen saturation. With the adjustment of the amount of the colorant group, it is possible to reproduce an absorption characteristic of hemoglobin having a predetermined concentration.

(2) Next, resin serving as a base is dissolved in a mixed solution obtained by dispersing the predetermined amount of the prepared colorant group for reproduction of the absorption characteristic of hemoglobin with the predetermined concentration in an organic solvent, for example, chlorinated hydrocarbon. At this time, an appropriate combination is selected in consideration of the solubility of the colorant and the base material. Examples of the chlorinated hydrocarbon include dichloromethane (CH₂Cl₂). Examples of the resin include acrylic resin.

(3) The solid sample 3 is prepared by volatilizing the organic solvent from the mixed solution in which the resin has been dissolved.

It is preferable that the colorant group thus prepared include at least a first colorant having two absorption peak wavelengths in a wavelength band where a wavelength is 520 to 600 nm, and a second colorant having one absorption peak wavelength in a wavelength band where a wavelength is 400 to 440 nm. As a result, it is possible to reproduce the absorption characteristic of hemoglobin having a strong absorption band called Q band derived from porphyrin in the vicinity of 550 nm to be described later.

FIG. 4 is a view for describing the calibration of the endoscope system using the solid sample 3. A distal end of an insertion tube 110 of the endoscope is brought close to the solid sample 3 to image the solid sample 3. Using the image data of this solid sample 3, the endoscope system creates a correspondence relationship between known hemoglobin concentration and hemoglobin oxygen saturation and parameters obtained from the image data. This point will be described in the following description on the endoscope system 10.

(Configuration of Endoscope System)

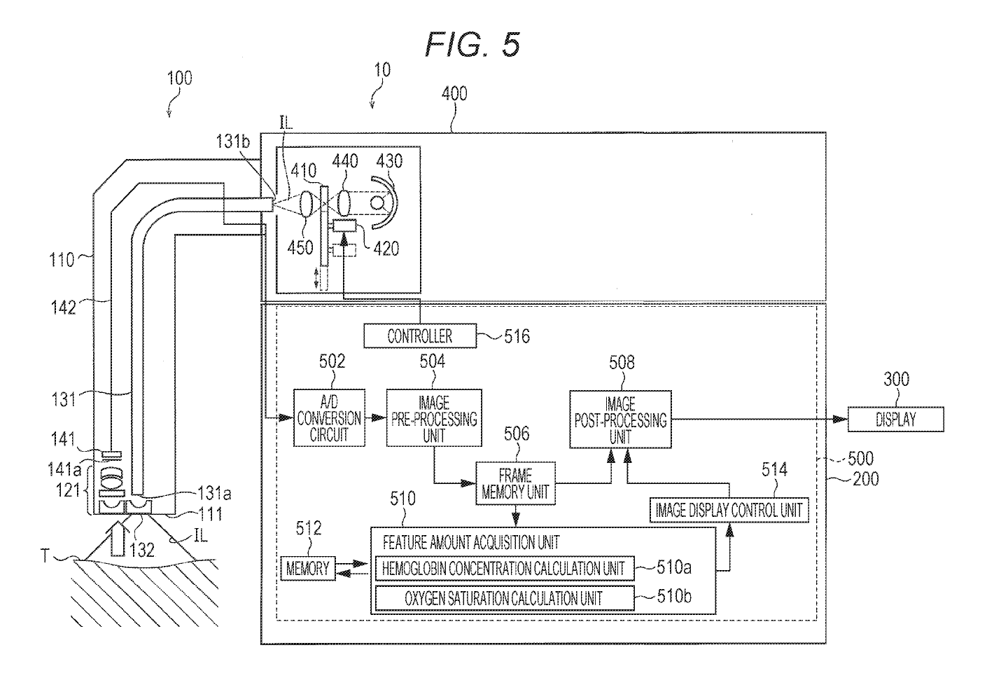

FIG. 5 is a block diagram illustrating a configuration of the endoscope system 10 used in the present embodiment. The endoscope system 10 includes an electronic endoscope (endoscope) 100, a processor 200, a display 300, and a light source device 400. The electronic endoscope 100 and the display 300 are detachably connected to the processor 200. The processor 200 includes an image processing unit 500. The light source device 400 is detachably connected to the processor 200.

The electronic endoscope 100 has the insertion tube 110 to be inserted into a body of a subject. Inside the insertion tube 110, a light guide 131 extending substantially over the entire length of the insertion tube 110 is provided. A distal end 131a which is one end of the light guide 131 is positioned at the distal end of the insertion tube 110, that is, in the vicinity of an insertion tube distal end 111, and a proximal end 131b which is the other end of the light guide 131 is positioned at a connection portion with the light source device 400. Therefore, the light guide 131 extends from the connection portion with the light source device 400 to the vicinity of the insertion tube distal end 111.

The light source device 400 includes, as a light source, a light source lamp 430 that generates light having a large amount of light such as a xenon lamp. The light emitted from the light source device 400 is incident on the proximal end 131b of the light guide 131 as illumination light IL. The light incident on the proximal end 131b of the light guide 131 is guided to the distal end 131a through the light guide 131 and emitted from the distal end 131a. At the insertion tube distal end 111 of the electronic endoscope 100, a light distribution lens 132 disposed so as to oppose the distal end 131a of the light guide 131 is provided. The illumination light IL emitted from the distal end 131a of the light guide 131 passes through the light distribution lens 132 and illuminates a living tissue T in the vicinity of the insertion tube distal end 111.

At the insertion tube distal end 111 of the electronic endoscope 100, an objective lens group 121 and an imaging element 141 are provided. The objective lens group 121 and the imaging element 141 form an imaging unit. Light reflected or scattered on a surface of the living tissue T out of the illumination light IL is incident on the objective lens group 121 and is condensed to form an image on a light-receiving surface of the imaging element 141. As the imaging element 141, it is possible to use a known imaging element such as a charge coupled device (CCD) image sensor for imaging a color image provided with a color filter 141a on a light-receiving surface thereof and a complementary metal oxide semiconductor (CMOS) image sensor.

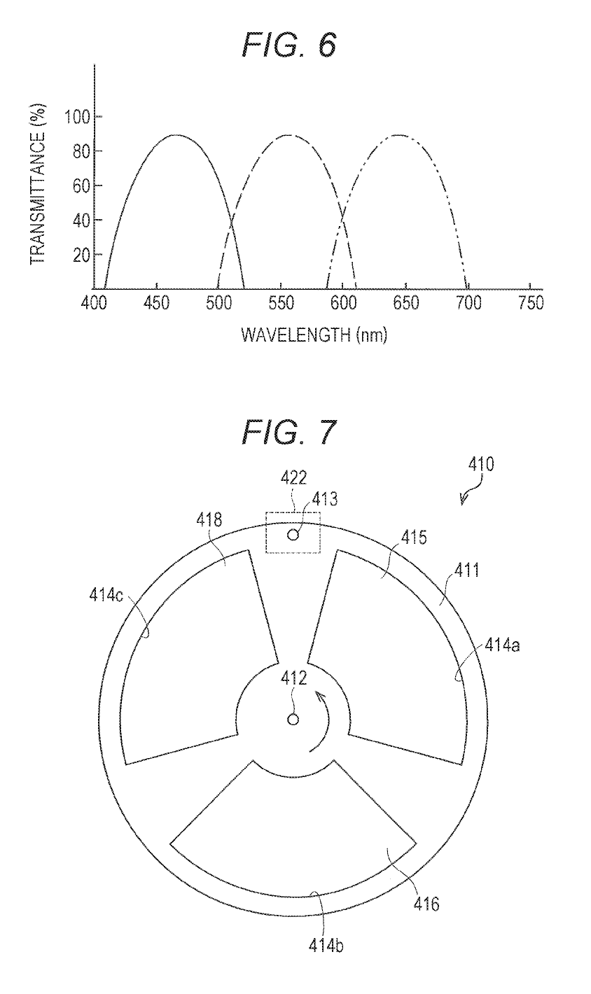

The color filter 141a is a so-called on-chip filter which is formed directly on each light-receiving element of the imaging element 141 and in which an R color filter that passes red light, a G color filter that passes green light, and a B color filters that passes blue light are arrayed. FIG. 6 is a graph illustrating an example of spectral characteristics of the respective filters of red (R), green (G) and blue (B) of the imaging element used in the present embodiment. The R color filter of the present embodiment is a filter that passes light having a wavelength longer than a wavelength of about 570 nm (for example, 580 nm to 700 nm), the G color filter is a filter that passes light having a wavelength of about 470 nm to 620 nm, and the B color filter is a filter that passes light having a wavelength shorter than a wavelength of about 530 nm (for example, 420 nm to 520 nm).

The imaging element 141 is an imaging means that images the living tissue T illuminated by each of a plurality of beams of lights, and generates color image data corresponding to each of the beams of light, and is an image data generation means that illuminates the living tissue T with a plurality of beams of light having different wavelength ranges to generate color image data corresponding to the light reflected or scattered on the living tissue T. The imaging element 141 is controlled to be driven in synchronization with the image processing unit 500 to be described later, and outputs color image data corresponding to an image of the living tissue T imaged on the light-receiving surface periodically (for example, at intervals of 1/30 seconds). The color image data output from the imaging element 141 is sent to the image processing unit 500 of the processor 200 via a cable 142.

The image processing unit 500 mainly includes an A/D conversion circuit 502, an image pre-processing unit 504, a frame memory unit 506, and an image post-processing unit 508, a feature amount acquisition unit 510, a memory 512, an image display control unit 514, and a controller 516.

The A/D conversion circuit 502 A/D converts the color image data input from the imaging element 141 of the electronic endoscope 100 via the cable 142 and outputs digital data. The digital data output from the A/D conversion circuit 502 is sent to the image pre-processing unit 504.

The image pre-processing unit 504 generates color image data of R, G, and B components constituting an image by demosaicing digital data from R digital image data imaged by a light-receiving element in the imaging element 141 to which the R color filter is mounted, G digital image data imaged by a light-receiving element in the imaging element 141 to which the G color filter is mounted, and B digital image data imaged by a light-receiving element in the imaging element 141 to which the B color filter is mounted. In addition, the image pre-processing unit 504 is a part that performs predetermined signal processing such as color correction, a matrix operation, and white balance correction on the generated R, G, B color image data.

The frame memory unit 506 temporarily stores the color image data of each image which has been imaged by the imaging element 141 and subjected to the signal processing.

The image post-processing unit 508 reads the color image data stored in the frame memory unit 506 or performs signal processing (y correction or the like) on image data generated by the image display control unit 514 to be described later, thereby generating screen data to be displayed on the display. The image data generated by the image display control unit 514 includes data of a distribution image of a feature amount such as an oxygen saturation distribution image illustrating a distribution of a hemoglobin oxygen saturation of the living tissue T as will be described later. The generated screen data (video format signal) is output to the display 300. As a result, the image of the living tissue T, the distribution image of the feature amount of the living tissue T, and the like are displayed on a screen of the display 300.

In response to an instruction from the controller 516, the feature amount acquisition unit 510 calculates a hemoglobin concentration and the hemoglobin oxygen saturation of the imaged living tissue T as feature amounts and generates distribution images of these features on the image of the imaged living tissue T, that is, a distribution image indicating a distribution of the hemoglobin concentration and the oxygen saturation distribution image indicating the distribution of the hemoglobin oxygen saturation as will be described later.

Since the feature amount is calculated by an operation using the color image data of the living tissue T illuminated with the plurality of beams of light having different wavelength ranges, the feature amount acquisition unit 510 calls out the color image data and various types of information used in the feature amount acquisition unit 510 from the frame memory unit 506 or the memory 512.

The image display control unit 514 performs control such that the hemoglobin oxygen saturation distribution image generated by the feature amount acquisition unit 510 is displayed in the state of being superimposed on the image of the imaged living tissue T.

The controller 516 is a part that performs not only an operation instruction and operation control of each part of the image processing unit 500 but also an operation instruction and operation control of each part of the electronic endoscope 100 including the light source device 400 and the imaging element 141.

Incidentally, the feature amount acquisition unit 510 and the image display control unit 514 may be configured by a software module that serves each of the above-described functions as a program is activated and executed on a computer, or may be configured by hardware.

In this manner, the processor 200 has both the function of processing the color image data output from the imaging element 141 of the electronic endoscope 100 and the function of instructing and controlling the operations of the electronic endoscope 100, the light source device 400, and the display 300.

The light source device 400 is a light-emitting means that emits first light, second light, and third light, and causes the first light, the second light, and the third light to be incident on the light guide 131. The light source device 400 of the present embodiment emits the first light, the second light, and the third light having different wavelength ranges, but may emit four or more beams of light. In this case, the fourth light may be light having the same wavelength range as that of the first light. The light source device 400 includes a condenser lens 440, a rotary filter 410, a filter control unit 420, and a condenser lens 450, in addition to the light source lamp 430. The light which is substantially parallel light and emitted from the light source lamp 430 is, for example, white light, is condensed by the condenser lens 440 and passes through the rotary filter 410, and then, is condensed again by the condenser lens 450, and is incident on the proximal end 131b of the light guide 131. Incidentally, the rotary filter 410 is movable between a position on an optical path of light radiated from the light source lamp 430 and a retreat position outside the optical path by a moving mechanism (not illustrated) such as a linear guideway. Since the rotary filter 410 includes a plurality of filters having different transmission characteristics, a wavelength range of light emitted from the light source device 400 differs depending on a type of the rotary filter 410 crossing the optical path of the light radiated from the light source lamp 430.

Incidentally, the configuration of the light source device 400 is not limited to that illustrated in FIG. 5.

For example, a lamp that generates convergent light instead of the parallel light to the light source lamp 430 may be adopted. In this case, for example, a configuration in which light radiated from the light source lamp 430 is converged in front of the condenser lens 440 and is made incident on the condenser lens 440 as diffused light may be adopted. In addition, a configuration in which substantially parallel beams of light, generated by the light source lamp 430, are directly incident on the rotary filter 410 may be adopted instead of using the condenser lens 440. In addition, in the case of using the lamp that generates the convergent light, a configuration in which a collimator lens is used instead of the condenser lens 440 to cause light to be incident on the rotary filter 410 in the state of substantially parallel beams of light may be adopted. For example, when an interference-type optical filter, such as a dielectric multilayer film filter, is used as the rotary filter 410, it is possible to obtain more favorable filter characteristics by causing substantially parallel beams of light to be incident on the rotary filter 410 to make incident angles of the beams of light with respect to the optical filter uniform. In addition, a lamp that generates divergent light may be adopted as the light source lamp 430. Even in this case, it is possible to adopt the configuration in which the collimator lens is used instead of the condenser lens 440 to cause substantially parallel beams of light to be incident on the rotary filter 410.

In addition, the light source device 400 is configured to emit the plurality of beams of light having different wavelength ranges by transmitting the light radiated from the single light source lamp 430 to the optical filter, but it is also possible to use a semiconductor light source, such as a light-emitting diode and a laser element that outputs a laser beam, that emit a plurality of different beams of light having different wavelength ranges, as a light source of the light source device 400, for example, instead of the light source lamp 430. In this case, the rotary filter 410 is not necessarily used. In addition, the light source device 400 can be also configured such that the light source device 400 separately emits, for example, combined white light including excitation light having a predetermined wavelength range and fluorescent light excited and emitted by the excitation light and light having a predetermined narrow wavelength range. The configuration of the light source device 400 is not particularly limited as long as the light source device 400 emits a plurality of beams of light having different wavelength ranges.

The rotary filter 410 is a disk-type optical unit having a plurality of optical filters, and is configured such that a passing wavelength range of light changes depending on a rotation angle thereof. The rotary filter 410 of the present embodiment includes three optical filters having different passing wavelength bands, but may have four, five, or six or more optical filters. The rotation angle of the rotary filter 410 is controlled by the filter control unit 420 connected to the controller 516. The controller 516 controls the rotation angle of the rotary filter 410 via the filter control unit 420, thereby switching a wavelength range of the illumination light IL supplied to the light guide 131 passing through the rotary filter 410.

FIG. 7 is an external view (front view) of the rotary filter 410. The rotary filter 410 includes a substantially discoid frame 411 and three fan-shaped optical filters 415, 416, and 418. Three fan-shaped windows 414a, 414b, and 414c are formed at equal intervals around a central axis of the frame 411, and the optical filters 415, 416, and 418 are fitted to the windows 414a, 414b, and 414c, respectively. Incidentally, all the optical filters of the present embodiment are the dielectric multilayer film filters, but other types of optical filters (for example, an absorption-type optical filter, an etalon filter using a dielectric multilayer film as a reflective film) may be used.

In addition, a boss hole 412 is formed on the central axis of the frame 411. An output shaft of a servomotor (not illustrated) provided in the filter control unit 420 is inserted and fixed in the boss hole 412, and the rotary filter 410 rotates together with the output shaft of the servo motor.

When the rotary filter 410 rotates in a direction indicated by an arrow in FIG. 7, the optical filter on which light is incident switches in the order of the optical filters 415, 416, and 418 so that a wavelength band of the illumination light IL passing through the rotary filter 410 is sequentially switched.

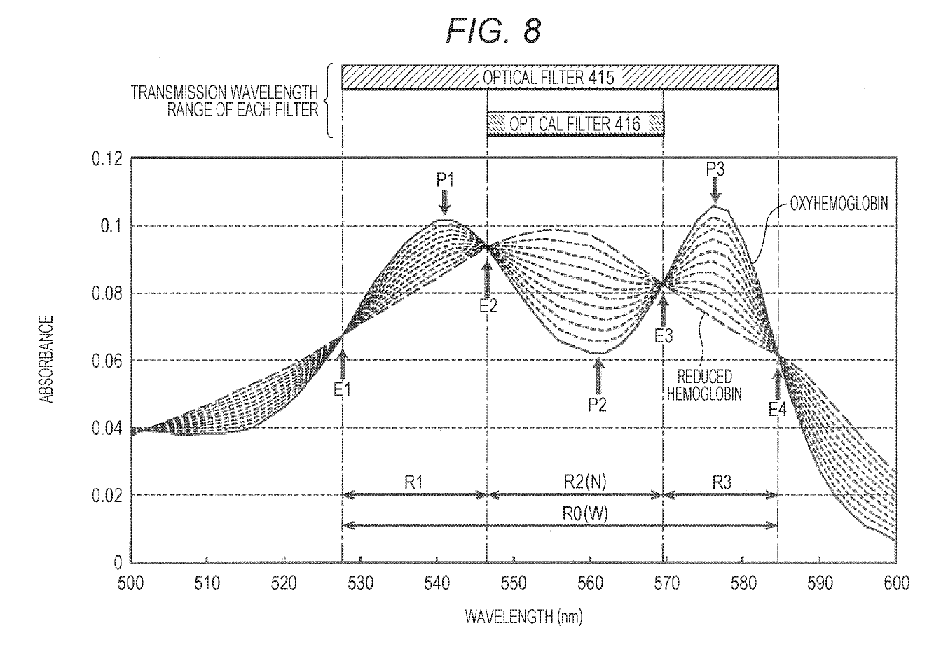

The optical filters 415 and 416 are optical band pass filters that selectively pass light in a band of 550 nm. As illustrated in FIG. 8, the optical filter 415 is configured to pass light in a wavelength range R0 (W band) from isosbestic points E1 to E4 with a low loss and block light in other wavelength ranges. In addition, the optical filter 416 is configured to pass light in a wavelength range R2 (N band) from isosbestic points E2 to E3 with a low loss and to block light in the other wavelength ranges.

In addition, the optical filter 418 is an ultraviolet cut filter, and the light radiated from the light source lamp 430 transmits through the optical filter 418 in the visible light wavelength range. The light transmitted through the optical filter 418 is used as white light WL for imaging of a normal observation image. Incidentally, it may be configured such that the window 414c of the frame 411 is open without using the optical filter 418.

Therefore, the light transmitted through the optical filter 415 out of the light radiated from the light source lamp 430 is hereinafter referred to as Wide light, the light transmitted through the optical filter 416 out of the light radiated from the light source lamp 430 is hereinafter referred to as Narrow light, and the light transmitted through the optical filter 418 out of the light emitted from the light source lamp 430 is hereinafter referred to as the white light WL.

FIG. 8 is a graph illustrating an example of the absorption spectrum of hemoglobin in the vicinity of 550 nm.

As illustrated in FIG. 8, a wavelength range R1 is a band including a peak wavelength of an absorption peak P1 derived from oxyhemoglobin, a wavelength range R2 is a band including a peak wavelength of an absorption peak P2 derived from reduced hemoglobin, and a wavelength range R3 is a band including a peak wavelength of an absorption peak P3 derived from oxyhemoglobin. In addition, the wavelength range R0 includes the respective peak wavelengths of the three absorption peaks P1, P2, and P3.

In addition, the wavelength range R0 of the optical filter 415 and the wavelength range R2 of the optical filter 416 are included in the passing wavelength range (FIG. 6) of the G color filter of the color filter 141a. Therefore, an image of the living tissue T formed by the light passing through the optical filter 415 or 416 is obtained as an image of the G component of the color image data imaged by the imaging element 141.

A through-hole 413 is formed in a peripheral edge portion of the frame 411. The through-hole 413 is formed at the same position (phase) as that of a boundary between the window 414a and the window 414c in a rotation direction of the frame 411. A photo-interrupter 422 configured to detect the through-hole 413 is arranged around the frame 411 so as to surround a part of the peripheral edge portion of the frame 411. The photo-interrupter 422 is connected to the filter control unit 420.

In this manner, the light source device 400 of the present embodiment is preferably configured so as to sequentially switch the plurality of optical filters 415, 416, and 418 in the optical path of the light radiated from the light source lamp 430 to emit the beams of light having different wavelength ranges, that is, the Wide light, the Narrow light, and the white light WL as the illumination light IL.

(Calculation of Feature Amount of Living Tissue)

The feature amount (the hemoglobin concentration or the hemoglobin oxygen saturation) of the living tissue T is calculated by the feature amount acquisition unit 510 of the processor 500. Hereinafter, a description will be given regarding a process of calculating the hemoglobin concentration and the hemoglobin oxygen saturation of the living tissue T from the image of the imaged living tissue T as the feature amounts.

As illustrated in FIG. 8, hemoglobin has a strong absorption band which is called Q band and derived from porphyrin in the vicinity of 550 nm. The absorption spectrum of hemoglobin changes according to an oxygen saturation that represents a proportion of oxyhemoglobin HbO in the total hemoglobin. A waveform of a solid line in FIG. 8 is an absorption spectrum where an oxygen saturation is 100%, that is, the oxyhemoglobin HbO, and a waveform of a long broken line is an absorption spectrum where an oxygen saturation is 0%, that is, reduced hemoglobin Hb. In addition, short broken lines indicate absorption spectrums of hemoglobin at intermediate oxygen saturations of 10, 20, 30, . . . , and 90%, that is, a mixture of the oxyhemoglobin HbO and the reduced hemoglobin Hb.

As illustrated in FIG. 8, the oxyhemoglobin HbO and the reduced hemoglobin Hb have peak wavelengths different from each other in the Q band. Specifically, the oxyhemoglobin HbO has the absorption peak P1 in the vicinity of a wavelength of 542 nm and the absorption peak P3 in the vicinity of a wavelength of 576 nm. On the other hand, the reduced hemoglobin Hb has the absorption peak P2 in the vicinity of 556 nm. FIG. 8 illustrates the absorption spectrum when the sum of the concentrations of the oxyhemoglobin HbO and the reduced hemoglobin Hb is constant, and thus, the isosbestic points E1, E2, E3, and E4 at which an absorbance becomes constant appear regardless of ratios of the oxyhemoglobin HbO and the reduced hemoglobin Hb, that is, the oxygen saturation. In the following description, a wavelength band sandwiched between the isosbestic points E1 and E2 is the wavelength band R1 described above with the optical filter 410, a wavelength range sandwiched between the isosbestic points E2 and E3 is the wavelength band R2, a wavelength band sandwiched between the isosbestic points E3 and E4 is the wavelength band R3, and a wavelength band sandwiched between the isosbestic points E1 and E4, that is, a combined band of the wavelength bands R1, R2, and R3 is the wavelength band R0. Therefore, a wavelength band of the Wide light, which is transmitted light that has been transmitted through the optical filter 415 out of the light radiated from the light source lamp 430, is the wavelength band R0, and a wavelength band of the Narrow light, which is transmitted light that has been transmitted through the optical filter 416 out of the light radiated from the light source lamp 430, is the wavelength band R2.

As illustrated in FIG. 8, the absorption of hemoglobin increases or decreases linearly relative to the oxygen saturation in the wavelength bands R1, R2, and R3. Specifically, total values AR1 and AR3 of the absorbances of hemoglobin in the wavelength bands R1 and R3 increase linearly relative to the concentration of oxyhemoglobin, that is, the oxygen saturation. In addition, a total value AR2 of absorbances of hemoglobin in the wavelength band R2 increases linearly relative to the concentration of reduced hemoglobin.

Here, the oxygen saturation is defined by the following Formula (1).

$$Formula ( 1 ) \text{:}$$

$$Sat = \frac{[ HbO ]}{[ Hb ] + [ HbO ]} \qquad [ Formula 1 ]$$

wherein,

Sat: Oxygen Saturation

[Hb]: Concentration of Reduced Hemoglobin

[HbO]: Concentration of Oxyhemoglobin

[Hb]+[HbO]: Hemoglobin concentration (tHb)

In addition, Formula (2) and Formula (3) expressing the concentrations of the oxyhemoglobin HbO and the reduced hemoglobin Hb are obtained from Formula (1).

Formula (2):

[HbO]=Sat·([Hb]+[HbO]) [Formula 2]

Formula (3):

[Hb]=(1−Sat)·([Hb]+[HbO]) [Formula 3]

Therefore, the total values AR1, AR2, and AR3 of the absorbances of hemoglobin become feature amounts that depend on both the oxygen saturation and the hemoglobin concentration.

Here, it has been found that a total value of absorbances in the wavelength band R0 does not depend on the oxygen saturation but is a value determined by the hemoglobin concentration. Therefore, the hemoglobin concentration can be quantified based on the total value of absorbances in the wavelength band R0. In addition, the oxygen saturation can be quantified based on the total value of absorbances in the wavelength band R1, the wavelength band R2, or the wavelength band R3 and the hemoglobin concentration quantified based on the total value of absorbances of the wavelength band R0.

The feature amount acquisition unit 510 of the present embodiment includes: a hemoglobin amount calculation unit 510a that calculates and acquires a hemoglobin concentration of the living tissue T based on a first ratio, which will be described later, sensitive to the hemoglobin concentration of the living tissue T; and an oxygen saturation calculation unit 510b that calculates and acquires a hemoglobin oxygen saturation of the living tissue T based on the calculated hemoglobin concentration and a second ratio, which will be described later, sensitive to the hemoglobin oxygen saturation. The fact that the first ratio is sensitive to the hemoglobin concentration means that the first ratio changes when the hemoglobin concentration changes. Similarly, the fact that the second ratio is sensitive to the hemoglobin concentration and the hemoglobin oxygen saturation means that the second ratio changes when the hemoglobin concentration and the hemoglobin oxygen saturation change.

A value of an luminance component of the color image data of the living tissue T illuminated with the Wide light (light in the wavelength band R0 transmitted through the optical filter 415) corresponds to (is reflected on) the total value of absorbances in the wavelength band R0 described above, and thus, the hemoglobin amount calculation unit 510a of the feature amount acquisition unit 510 of the present embodiment calculates the hemoglobin concentration based on the luminance component of the color image data in the wavelength band R0. Here, the luminance component is obtained by multiplying the R component of the color image data by a predetermined coefficient, multiplying the G component of the color image data by a predetermined coefficient, multiplying a value of the B component of the color image data by a predetermined coefficient, and summing up these multiplication results.

Specifically, the hemoglobin amount calculation unit 510a of the feature amount acquisition unit 510 calculates the hemoglobin concentration based on a ratio Wide/WL(R), obtained by dividing a luminance component Wide (hereinafter, also simply referred to as Wide) of color image data (second color image data) of the living tissue T obtained using the Wide light (second light) as the illumination light IL by an R component WL(R) of color image data (first color image data) of the living tissue T obtained using the white light WL (first light) as the illumination light IL or a total component WL(R)+WL(G) of the R component WL(R) and a G component WL(G), or based on Wide/{WL(R)+WL(G)} (the first ratio). The use of the ratio Wide/WL(R) or Wide/{WL(R)+WL(G)} obtained by dividing the luminance component Wide by WL(R) or {WL(R)+WL(G)} in the calculation of the hemoglobin concentration is a condition that can minimize influence of the scattering of the living body on spectral information of blood in the present system. In particular, a reflection spectrum of the living tissue T, such as an inner wall of a digestive tract, is easily affected by wavelength characteristics of scattering of illumination light by the living tissue T in addition to wavelength characteristics of absorption by components constituting the living tissue T (specifically, absorption spectrum characteristics of oxyhemoglobin and reduced hemoglobin). The R component WL(R) of the color image data (first color image data) of the living tissue T obtained using the white light WL (first light) as the illumination light IL or the total component WL(R)+WL(G) of the R component and the G component indicates the degree of scattering of the illumination light IL in the living tissue T without being affected by the hemoglobin concentration and hemoglobin oxygen saturation. Therefore, in order to remove the influence of scattering in the living tissue T of the illumination light IL from the reflection spectrum of the living tissue T, a wavelength band of the white light WL (reference light) is preferably set to include a wavelength band in which one of components of the first color image data is insensitive to a change in hemoglobin concentration of the living tissue T. Further, the wavelength band of the white light WL (reference light) is preferably set to include a wavelength band in which one of the components of the first color image data is insensitive to a change in oxygen saturation.

In the present embodiment, a reference table, which indicates a correspondence relationship between information of the above-described first ratio and the hemoglobin concentration in the above-described solid sample 3 reproducing the absorption characteristic of hemoglobin with the predetermined concentration, is stored in advance in the memory 512, and the hemoglobin amount calculation unit 510a of the feature amount acquisition unit 510 uses this reference table to calculate a hemoglobin concentration based on a value of the first ratio in the captured color image data of the living tissue T.

Although the ratio Wide/WL(R) between the luminance component Wide of the color image data (second color image data) of the living tissue T obtained using the Wide light (second light) as the illumination light IL and the R component WL(R) of the color image data (first color image data) of the living tissue T obtained using the white light WL (first light) as the illumination light IL or the total component WL(R)+WL(G) of the R component WL(R) and the G component WL(G), or Wide/{WL(R)+WL(G)} can be used as the first ratio in the calculation of the hemoglobin concentration in the present embodiment, this value is desirably optimized by wavelength characteristics of a filter to be used.

Further, the total value of absorbances in the wavelength band R2 decreases along with the increase of the oxygen saturation, and the total value of absorbances in the wavelength band R0 changes depending on the hemoglobin concentration but is constant regardless of the change of the oxygen saturation as described above. Thus, the oxygen saturation calculation unit 510b of the feature amount acquisition unit 510 calculates the oxygen saturation based on a second ratio to be defined below. That is, the oxygen saturation calculation unit 510b of the feature amount acquisition unit 510 calculates a ratio Narrow/Wide, as the second ratio, between a luminance component Narrow (hereinafter, also simply referred to as Narrow) of color image data (third color image data) of the living tissue T illuminated with the Narrow light, which is the light of the wavelength band R2 that has passed through the optical filter 416, and the luminance component Wide of the color image data (second color image data) of the living tissue T illuminated with the Wide light (light of the wavelength band R0 transmitted through the optical filter 415). On the other hand, a correspondence relationship indicating a relationship between the hemoglobin concentration and a lower limit of the second ratio at the oxygen saturation=0% and an upper limit of the second ratio Narrow/Wide at the oxygen saturation=100% is obtained from the solid sample 3 described above, and stored in memory 512 in advance. The oxygen saturation calculation unit 510b of the feature amount acquisition unit 510 uses a calculation result of the hemoglobin concentration obtained from the color image data generated by imaging the living tissue T and the above correspondence relationship to obtain the lower limit value and the upper limit value of the second ratio. The lower limit value and the upper limit value are values corresponding to the oxygen saturations 0% and 100%, respectively. Further, the oxygen saturation calculation unit 510b calculates the oxygen saturation based on any position in a range between the lower limit value and the upper limit value corresponding to the oxygen saturation 0 to 100% where the value of the second ratio Narrow/Wide of the imaged living tissue T is present by utilizing the fact that the second ratio changes linearly depending on the oxygen saturation between the obtained lower limit value and upper limit value. In this manner, the oxygen saturation calculation unit 510b of the feature amount acquisition unit 510 calculates the oxygen saturation.

In addition, a reference table, which indicates a correspondence relationship between the hemoglobin concentration and the value of the second ratio, and the hemoglobin oxygen saturation, is obtained from the solid sample 3 described above and stored in the memory 512 in advance, and the hemoglobin oxygen saturation can also be calculated from the calculated second ratio with reference to this reference table.

Although the second ratio is used as the ratio between the luminance component Narrow of the color image data (third color image data) of the living tissue T illuminated with the Narrow light and the luminance component Wide of the color image data (second color image data) of the living tissue T illuminated with the Wide light in the present embodiment, it is also possible to use a ratio between a G component Narrow(G) of the color image data (third color image data) of the living tissue T illuminated with the Narrow light and a G component Wide(G) of the color image data (second color image data) of the living tissue T illuminated with the Wide light.

In addition, the Narrow light in the wavelength band R2 is used to illuminate the living tissue T in order for the calculation of the second ratio in the present embodiment, but the light to be used is not limited to the Narrow light. For example, it is also possible to use light whose wavelength band is the wavelength band R1 or the wavelength band R2 with an intention of use of the wavelength band R1 or the wavelength band R2 where the total value of absorbances changes with the change of the oxygen saturation. In this case, a filter characteristic of the optical filter 416 may be set to the wavelength band R1 or the wavelength band R2.

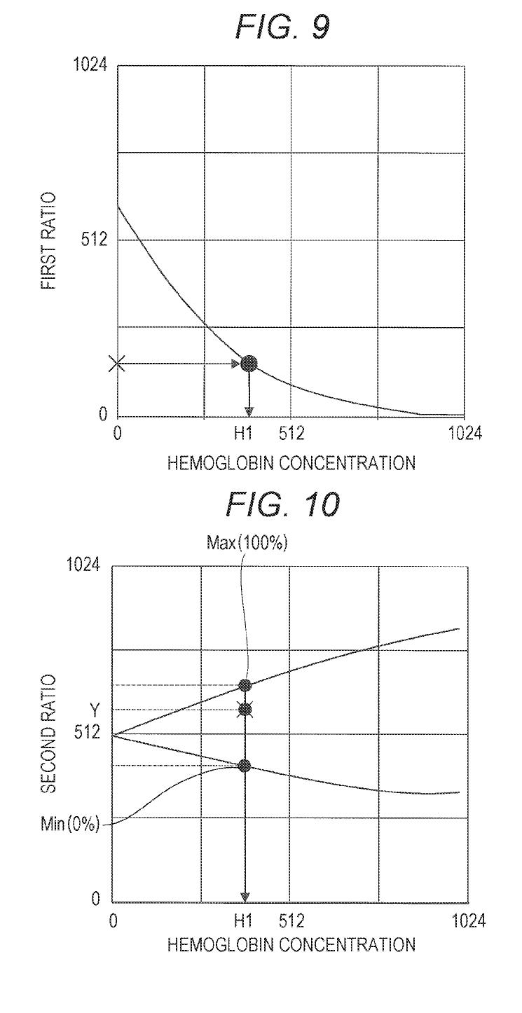

FIG. 9 is a graph illustrating an example of a relationship between the first ratio and the hemoglobin concentration. When obtaining the first ratio as described above, the hemoglobin amount calculation unit 510a of the feature amount acquisition unit 510 refers to a reference table indicating the correspondence relationship as illustrated in FIG. 9 to obtain the hemoglobin concentration based on the determined first ratio. FIG. 9 indicates that a concentration H1 of hemoglobin has been obtained based on a value of the first ratio. The numerical values on the horizontal and vertical axes in FIG. 9 are indicated by values of 0 to 1024 for convenience.

FIG. 10 is a graph illustrating an example of a relationship between the upper limit value and the lower limit value of the second ratio, and the hemoglobin concentration. Numerical values on the horizontal and vertical axes in FIG. 10 are indicated by values of 0 to 1024 for convenience.

When obtaining the second ratio as described above, the oxygen saturation calculation unit 510b of the feature amount acquisition unit 510 obtains the upper limit value and the lower limit value of the second ratio in the obtained hemoglobin concentration using the correspondence relationship illustrated in FIG. 10 based on the hemoglobin concentration and the second ratio obtained by the hemoglobin amount calculation unit 510a. The upper limit value indicates the oxygen saturation=100%, and the lower limit value indicates the oxygen saturation=0%. The oxygen saturation calculation unit 510b obtains a value of the oxygen saturation by obtaining any position between the upper limit and the lower limit where the second ratio is present. In FIG. 10, an upper limit value Max (100%) and a lower limit value Min (0%) at the concentration H1 of hemoglobin when a value of the second ratio is R2 are obtained. The value of the oxygen saturation is obtained based on the upper limit value Max (100%), the lower limit value Min (0%), and a value Y of the second ratio.

In this endoscope system 10, the correspondence relationships as illustrated in FIGS. 9 and 10 are created in advance (calibration is performed) in order to calculate the hemoglobin concentration and the hemoglobin oxygen saturation. In the present embodiment, the solid sample 3 is used to create these correspondence relationships.

Therefore, the memory 512 of the processor 200 stores a first correspondence relationship between the hemoglobin concentration and a value of the ratio Wide/WL(R) or Wide/{WL(R)+WL(G)} generated from a measurement result measured using the solid sample 3 as a calibration reference sample for the calculation of the hemoglobin oxygen saturation, and a second correspondence relationship between the hemoglobin oxygen saturation and the value of the ratio Narrow/Wide. Specifically, the first correspondence relationship includes an association between a calibration measurement value of the ratio Wide/WL(R) or Wide/{WL(R)+WL(G)} (the first ratio), which is a measurement result obtained by imaging the solid sample 3 with the electronic endoscope 100 as the calibration reference sample for the calculation of the hemoglobin oxygen saturation, and information on the hemoglobin concentration defined in solid sample 3. The second correspondence relationship includes an association between a calibration measurement value of the ratio Narrow/Wide, which is a measurement result obtained by imaging the solid sample 3 with the electronic endoscope 100 as the calibration reference sample, and information on the hemoglobin oxygen saturation defined in solid sample 3.

The processor 200 is configured to calculate the hemoglobin concentration and the hemoglobin oxygen saturation of the living tissue T using the stored first correspondence relationship and second correspondence relationship.

In this endoscope system 10, the following calibration using the solid sample 3 can be performed.

(1) As illustrated in FIG. 4, the above-described solid sample 3 is imaged by the electronic endoscope 100 to acquire each of the calibration measurement value of the ratio Wide/WL(R) or Wide/{WL(R)+WL(G)} and the calibration measurement value of the ratio Narrow/Wide.

(2) The processor 200 generates the first correspondence relationship between the hemoglobin concentration and the value of the ratio Wide/WL(R) or Wide/{WL(R)+WL(G)} including a first association between the calibration measurement value of the ratio Wide/WL(R) or Wide/{WL(R)+WL(G)} and the information on the hemoglobin concentration defined in the solid sample 3. Further, the processor 200 generates the second correspondence relationship between the hemoglobin oxygen saturation and the value of the ratio Narrow/Wide including a second association between the calibration measurement value of the ratio Narrow/Wide and the information on the hemoglobin oxygen saturation defined in the solid sample 3.

(3) The processor 200 stores the first correspondence relationship and the second correspondence relationship in the memory 512 in order to use the generated first correspondence relationship and second correspondence relationship in the calculation of the hemoglobin concentration and the hemoglobin oxygen saturation in the living tissue.

When performing the calibration using the solid sample 3 in the endoscope system 10, it is preferable that a plurality of types of solid samples having different contents of the colorant group corresponding to a plurality of concentrations of hemoglobin be prepared as the solid sample 3 and that the calibration measurement value of the ratio Wide/WL(R) or Wide/{WL(R)+WL(G)} and the calibration measurement value of the ratio Narrow/Wide be measurement results obtained by imaging each of the plurality of types of solid samples with the electronic endoscope 100 as reference samples. Since a plurality of calibration measurement values are obtained using the solid samples made of stable non-biological substances, it is possible to perform stable calibration.

In addition, when performing the calibration using the solid sample 3, it is preferable that a plurality of types of solid samples having different contents of the colorant group corresponding to a plurality of oxygen saturations be prepared as the solid sample 3 and that the calibration measurement value of the ratio Wide/WL(R) or Wide/{WL(R)+WL(G)} and the calibration measurement value of the ratio Narrow/Wide be measurement results obtained by imaging each of the plurality of types of solid samples with the electronic endoscope 100 as reference samples.

The ratio Wide/WL(R) or Wide/{WL(R)+WL(G)} is a ratio that is sensitive to the hemoglobin concentration of the living tissue, the ratio Narrow/Wide is a ratio sensitive to the hemoglobin oxygen saturation of the living tissue, the luminance component Wide is the component of the wavelength band in the range of 500 nm to 600 nm, and the luminance component Narrow is the component of the wavelength band narrower than the above-described wavelength band in the range of 500 nm to 600 nm. As a result, the hemoglobin concentration and the hemoglobin oxygen saturation can be accurately obtained.

Incidentally, according to the above embodiment, the processor 200 corrects the first correspondence relationship and the second correspondence relationship, created using the reference sample having the predetermined hemoglobin concentration and the predetermined hemoglobin oxygen saturation at the time of completion of the endoscope system 10 and recorded and held in the endoscope system, to match the first correspondence relationship and the second correspondence relationship obtained by imaging the solid sample 3 with the electronic endoscope 100.

However, according to another embodiment, it is also preferable that the processor 200 correct values of the ratio Wide/WL(R) or Wide/{WL(R)+WL(G)} and the ratio Narrow/Wide, acquired by imaging the living tissue T with the electronic endoscope 100, using correction coefficients without correcting the first correspondence relationship and the second correspondence relationship created using the reference sample having the predetermined hemoglobin concentration and the predetermined hemoglobin oxygen saturation at the time of completion of the endoscope system 10 and recorded and held in the endoscope system. In this case, the processor 200 stores, in the memory 412, the correction coefficients which allow the calibration measurement value of the ratio Wide/WL(R) or Wide/{WL(R)+WL(G)} (the calibration measurement value of the first ratio), which is the measurement result obtained by imaging the solid sample 3 with the electronic endoscope 100 as the calibration reference sample for the calculation of the hemoglobin oxygen saturation and the calibration measurement value of the ratio Narrow/Wide (the calibration measurement value of the second ratio) to be corrected to preset values, respectively. The processor 200 refers to the stored and held first correspondence relationship and second correspondence relationship using values obtained by correcting the first ratio obtained using values of image data of the imaged image of the living tissue T, specifically, the value of the ratio Wide/WL(R) or Wide/{WL(R)+WL(G)}, and the second ratio, specifically the value of the ratio Narrow/Wide using the above-described correction coefficients, thereby calculating the hemoglobin concentration and the hemoglobin oxygen saturation in the living tissue. The correction may be performed by, for example, multiplying or dividing the value of the ratio Wide/WL(R) or Wide/(WL(R)+WL(G)) and the second ratio, specifically, the value of the ratio Narrow/Wide by the correction coefficients, respectively.

In this case, the endoscope system 10 can perform the following calibration using the solid sample 3.

The processor 200 stores and holds the first correspondence relationship and the second correspondence relationship created using the reference sample having the predetermined hemoglobin concentration and the predetermined hemoglobin oxygen saturation at the time of completion of the endoscope system 10.

When performing the calibration, (1) the solid sample 3 is imaged by the electronic endoscope 100 to acquire each of the calibration measurement value of the ratio Wide/WL(R) or Wide/{(WL(R)+WL(G)} (the calibration measurement value of the first ratio) and the calibration measurement value of the ratio Narrow/Wide (the calibration measurement value of the second ratio).

(2) Next, the processor 200 calculates the correction coefficients which allow the calibration measurement value of the ratio Wide/WL(R) or Wide/{WL(R)+WL(G)} and the calibration measurement value of the ratio Narrow/Wide to be corrected to the preset values, respectively.

(3) The processor 200 stores the correction coefficients in the memory 512 in order to use the calculated correction coefficients to calculate the hemoglobin concentration and the hemoglobin oxygen saturation in the living tissue T by correcting the ratio Wide/WL(R) or Wide/{WL(R)+WL(G)} and the ratio Narrow/Wide obtained by imaging the living tissue T using the correction coefficients, respectively.

Incidentally, the solid sample 3 is imaged using the electronic endoscope 100, and thus, it is important to be capable of obtaining a calibration measurement value with a small variation depending on a place regardless of which part of the solid sample 3 is imaged. Thus, it is preferable that a variation of concentration of the colorant group in the solid sample 3 depending on a place be small. In this case, a variation of an average absorbance of the solid sample 3 in the wavelength band of 520 to 600 nm depending on a place is preferably 0 to 5% or less of an average value of the average absorbances for the place. The solid sample 3 can be realized by mixed dispersing the resin and the colorant group when the resin and the colorant group are dispersed in the organic solvent to form the mixed solution in the preparation method of the solid sample 3 described above.

Further, since the solid sample 3 is imaged using the electronic endoscope 100, it is important to obtain the spectral waveform of the absorbance as illustrated in FIG. 2, specifically, a calibration measurement value with a small variation in an average value of absorbances in the wavelength range X (500 nm to 600 nm) including the two absorption peaks regardless of which part of the solid sample 3 is imaged. Thus, it is preferable that a variation of concentrations between the colorant groups in the solid sample 3 depending on a place be small. Thus, a variation depending on a place of a ratio of an average absorbance in the wavelength range of 546 to 570 nm relative to the average absorbance in the wavelength band of 528 to 584 nm of the solid sample 3 is preferably 0 to 1% or less of an average value of the ratios for the place. The solid sample 3 can be realized by mixed dispersing each colorant in the organic solvent when the resin and the colorant group are dispersed in the organic solvent to form the mixed solution in the preparation method of the solid sample 3 described above.

Although the present embodiment has been described as above, the present invention is not limited to the above embodiment, and various modifications can be made within a scope of a technical idea of the present invention.

### REFERENCE SIGNS LIST

1 calibration sample
2 base
3 solid sample
10 endoscope system
100 electronic endoscope
110 insertion tube
111 insertion tube distal end
121 objective lens group
131 light guide
131a distal end
131b proximal end
132 lens
141 imaging element
141a color filter
142 cable
200 processor
300 display
400 light source unit
410 rotary filter
420 filter control unit
430 light source lamp
440 condenser lens
450 condenser lens
500 image processing unit
502 A/D conversion circuit
504 image pre-processing unit
506 frame memory unit
508 image post-processing unit
510 feature amount acquisition unit
512 memory
514 image display control unit
516 controller

1. A solid sample to be used as a calibration reference sample to calculate a hemoglobin concentration and a hemoglobin oxygen saturation in a living tissue, the solid sample comprising:
a colorant group made of non-biological substances which have a plurality of colorants and reproduce absorption characteristics of the hemoglobin with a predetermined concentration and a predetermined oxygen saturation by adjusting a mixing ratio of the plurality of colorants; and
a resin material in which each colorant of the colorant group is dispersed.

2. The solid sample according to claim 1, wherein
the colorant group includes at least a first colorant having two absorption peak wavelengths in a wavelength band where a wavelength is 520 to 600 nm, and a second colorant having one absorption peak wavelength in a wavelength band where a wavelength is 400 to 440 nm, and
a wavelength band of the absorption characteristics reproduced by the colorant group is a wavelength band of 400 to 600 nm.

3. The solid sample according to claim 1, wherein
an absorption spectrum of the wavelength band where the wavelength is 520 to 600 nm in the solid sample has two absorption peaks and an absorption bottom which is sandwiched between the two absorption peaks and has a lowest absorbance between the two absorption peaks,
each wavelength deviation between each of the two absorption peaks and each of corresponding absorption peaks of the hemoglobin, which respectively correspond to the two absorption peaks, is 2 nm or less,
a wavelength deviation between the absorption bottom and a corresponding absorption bottom of the hemoglobin, which corresponds to the absorption bottom, is 2 nm or less, and
each absorbance at each of the two absorption peaks is in a range of 95% to 105% with respect to each absorbance at each of the corresponding absorption peaks of the hemoglobin which respectively correspond to the two absorption peaks.

4. The solid sample according to claim 1, wherein
an absorption spectrum of the wavelength band where the wavelength is 520 to 600 nm in the solid sample has one absorption peak in a range of 546 to 570 nm, and
an absorbance at the absorption peak is in a range of 95% to 105% with respect to an absorbance at a corresponding absorption peak of the hemoglobin which corresponds to the absorption peak.

5. The solid sample according to claim 1, wherein
a variation depending on a place of the solid sample, of an average absorbance of the solid sample in the wavelength band where the wavelength is 520 to 600 nm of the solid sample is 5% or less of an average value of the average absorbances for the place.

6. The solid sample according to claim 1, wherein
a variation depending on a place of the solid sample, of a ratio of an average absorbance in a wavelength band where a wavelength is 546 to 570 nm relative to an average absorbance in a wavelength band where a wavelength is 528 to 584 nm of the solid sample is 1% or less of an average value of the ratios for the place.

7. An endoscope system comprising:
an endoscope including an imaging unit which has an imaging element configured to generate a plurality of pieces of image data by imaging a living tissue; and
a processor configured to calculate values of a first ratio and a second ratio between predetermined components using values of the components out of components of the plurality of pieces of image data and to calculate a hemoglobin concentration and a hemoglobin oxygen saturation in the living tissue using the values of the first ratio and the second ratio,
wherein the processor includes a storage unit storing a first correspondence relationship between the hemoglobin concentration and the value of the first ratio, the first correspondence relationship including an association between a calibration measurement value of the first ratio, which is a measurement result obtained by imaging the solid sample according to claim 1 with the endoscope as a calibration reference sample for calculation of the hemoglobin oxygen saturation, and information on the predetermined hemoglobin concentration of the solid sample, and a second correspondence relationship between the hemoglobin oxygen saturation and the value of the second ratio, the second correspondence relationship including an association between a calibration measurement value of the second ratio, which is a measurement result obtained by imaging the solid sample according to claim 1 with the endoscope, as the calibration reference sample, and information on the predetermined hemoglobin oxygen saturation of the solid sample, and
the processor is configured to calculate the hemoglobin concentration and the hemoglobin oxygen saturation in the living tissue using the first correspondence relationship and the second correspondence relationship.

8. An endoscope system comprising:
an endoscope including an imaging unit which has an imaging element configured to generate a plurality of pieces of image data by imaging a living tissue; and
a processor configured to calculate values of a first ratio and a second ratio between predetermined components using values of the components out of components of the plurality of pieces of image data and to calculate a hemoglobin concentration and a hemoglobin oxygen saturation in the living tissue using the values of the first ratio and the second ratio,
wherein the processor includes a storage unit storing a first correspondence relationship between the hemoglobin concentration and the value of the first ratio, a second correspondence relationship between the hemoglobin oxygen saturation and the value of the second ratio, and correction coefficients which allow a calibration measurement value of the first ratio and a calibration measurement value of the second ratio, which are measurement results obtained by imaging the solid sample according to claim 1, as a calibration reference sample for calculation of the hemoglobin oxygen saturation, with the endoscope, to be corrected to preset values, respectively, and
the processor is configured to refer to the first correspondence relationship and the second correspondence relationship using the values, obtained by correcting the values of the first ratio and the second ratio obtained using a value of the image data, using the correction coefficients to calculate the hemoglobin concentration and the hemoglobin oxygen saturation in the living tissue.

9. The endoscope system according to claim 7, wherein
the calibration measurement value of the first ratio and the calibration measurement value of the second ratio are measurement results obtained by imaging each of a plurality of types of solid samples having different contents of the colorant group corresponding to a plurality of hemoglobin concentrations, as the reference sample, with the endoscope.

10. The endoscope system according to claim 7, wherein
the first ratio is a ratio sensitive to the hemoglobin concentration of the living tissue,
the second ratio is a ratio sensitive to the hemoglobin oxygen saturation of the living tissue,
one of the components of the image data used for calculation of the first ratio is a component of a first wavelength band within a range of 500 nm to 600 nm, and
one of the components of the image data used for calculation of the second ratio is a component of a second wavelength band narrower than the first wavelength band.

11. A preparation method of a solid sample made of a non-biological substance to be used as a calibration reference sample to calculate a hemoglobin oxygen saturation, the preparation method comprising:
preparing a colorant group reproducing an absorption characteristic of hemoglobin having a predetermined hemoglobin oxygen saturation;
dissolving resin as a base material in a mixed solution in which a predetermined amount of the colorant group for reproduction of an absorption characteristic of hemoglobin with a predetermined concentration is dispersed in an organic solvent; and
volatilizing the organic solvent from the mixed solution in which the resin has been dissolved to prepare the solid sample.

12. The preparation method of a solid sample according to claim 11, wherein
the colorant group includes at least a first colorant having two absorption peak wavelengths in a wavelength band where a wavelength is 520 to 600 nm, and a second colorant having one absorption peak wavelength in a wavelength band where a wavelength is 400 to 440 nm.
